(19) United States
(12) Patent Application Publication
(10) Pub. No.: US 2010/0059358 A1
(43) Pub. Date: Mar. 11, 2010

(54) POTABLE WATER DISTILLER

(75) Inventors: Jonathan Ritchey, Kelowna (CA); Richard Weisbeck, Kelowna (CA)

Correspondence Address: SCHWABE, WILLIAMSON & WYATT, P.C., 1420 FIFTH, SUITE 3010, SEATTLE, WA, 98101, US

(73) Assignee: FREEDOM WATER COMPANY LTD., Kelowna, BC (CA)

(21) Appl. No.: 12/596,816

(22) PCT Filed: Apr. 21, 2008
(86) PCT No.: PCT/CA08/00756

§ 371 Date: Oct. 20, 2009

Related U.S. Application Data
(60) Provisional application No. 60/913,006, filed on Apr. 20, 2007.

(51) Int. Cl. C02F 1/04

(52) U.S. Cl. 203/10; 202/167

(57) ABSTRACT

A distiller to be used for water purification, said distiller comprising a condenser, an evaporation pan, means for allowing heated air to enter below said pan and joining an airflow above the pan, and an evaporator for collecting water from the airflow The method for purifying water using this distiller is to draw airflow above the evaporation pan containing contaminated water, passing the airflow across a cooling element, into a heat pipe, through a condenser and through the evaporation pan Purified water is collected from the airflow when it is passed across the cooling element.

This application claims the benefit of U.S. Provisional Patent Application No. 60/913,006 filed Apr. 20, 2007, which is hereby incorporated by reference.

### FIELD OF THE INVENTION

This invention relates to water production, and more particularly to the production of distilled water from the atmosphere.

### BACKGROUND OF THE INVENTION

At any given moment the earth's atmosphere contains approximately 326 million cubic miles of water and of this water, about 97% is saltwater and only about 3% is fresh water. Of the 3% of the atmospheric water that is fresh water, about 70% is frozen in Antarctica and of the remaining 30% only about 0.7% is available in liquid form. Atmospheric air thus contains about 0.16% of this 0.7%, or about 4,000 cubic miles of water, which is about eight times the amount of liquid water found in all the rivers of the world. Of that 0.7%, approximately:
0.16% of the 0.7% is found in the atmosphere;
0.8% of that 0.7% is found in soil moisture;
1.4% of that 0.7% is found in lakes; and
97.5% of that 0.7% is found in groundwater.

This ratio is maintained by acceleration or retardation of the rates of evaporation and condensation, irrespective of the activities of man. For most life forms on Earth, the liquid water is the sole source and means of regenerating wholesome water.

Currently, about 1.2 billion people lack access to safe drinking water and that number is increasing steadily with forecasts of a potential 2.3 billion, or one-third of the earth's population, without access to safe water by 2025 (World Health Organization's statistics from World Commission on Water for the 21st Century). These at-risk children and their families are not restricted to rural areas in undeveloped nations. “Millions of poor urban dwellers have been left without water supply and sanitation in the rapidly growing cities of the developing world. The poor are often forced to pay exorbitant prices for untreated water, much of it deadly,” reports William Cosgrove, director of World Water Vision, Paris.

A rapid increase in water demand, particularly for industrial and household use, is being driven by population growth and socioeconomic development. If this growth trend continues, consumption of water by the industrial sector will be double by 2025 (WMO).

Urban population growth will increase demand for household water, but poorly planned water and sanitation services will lead to a breakdown in services for hundreds of millions of people. Many households will remain unconnected to clean piped water.

There is therefore a global need for cost effective and scalable sources of potable water. Current technologies to obtain potable water require significant energy to operate efficiently and the resultant cost of treated water puts these technologies out of reach for the majority in need. Desalination plants exist in rich nations such as the United States and Saudi Arabia but are not feasible everywhere. The lack of infrastructure in developing nations makes large desalination plants with high-volume production impractical, as there is no way to transport the water efficiently.

### SUMMARY OF THE INVENTION

There is a need for small scalable water distillers that meet the needs of individuals, communities and industries. The distiller according to the invention responds to that need by including a water extraction unit that can function on or off the traditional water grid to make clean pure water, wherever the need exists.

The present invention is a distiller that extracts pure water from literally any source of water regardless of whether the water source is salt water or highly contaminated water. The distiller may utilize the sun as the primary energy source thereby eliminating the need for costly fuels, hydro or battery power sources.

The distiller according to the invention provides for the creation of pure water for virtually any application. Private individuals, industries and communities may control their own water supply through the use of this technology. It is practical for many uses in domestic, commercial or military applications and offers ease of use and clean water of a highest quality anywhere, anytime. The modular design of the distiller allows for increased capacity, simply by adding more modules. The distiller is scalable and may be constructed to suit particular applications and available resources.

The distiller may be applied to a variety of uses including residential, recreational, commercial, agricultural, military and life saving in water deprived regions of the world.

The distiller according to the invention may be used for obtaining pure drinking water, for cooking purposes, or for other household uses such as cleaning or bathing. The distiller may also be used on boats or in vacation areas, on camping trips, trekking, and places where drinking water delivery systems are not developed. The distiller may be used to produce fresh water for bottling purposes or for larger commercial applications such as restaurants, offices, schools, hotel lobbies, cruise ships, hospitals and other public buildings. The distiller may also be used in playing fields and sports arenas.

Additionally, the distiller according to the invention may be used to augment the supply of water being used to irrigate selected crops using micro or drip irrigation systems. Such systems are designed to deliver the appropriate amount of water at the appropriate time, directly to the roots of plants. As well, the distiller may be used to for bottled water production or virtually any other application where water is needed.

The distiller according to the invention provides an opportunity to end much suffering. The death and misery that flow from unsafe water is overwhelming. More than 5,000 children die daily from diseases caused by consuming water and food contaminated with bacteria, according to a recent study released by UNICEF, the World Health Organization (WHO) and the UN Environment Program (UNEP).

The distiller according to the invention offers a practical and affordable solution to many of the world's water supply problems.

In summary, the distiller is a device that utilizes various input source energy supplies to create an internal evaporation and condensation process that extracts a pure water source from any existing saline or contaminated water source.

The distiller may be powered by a 12 Volt compressor that allows for an efficient condensation process for creating a potable water supply, and that may be portable, allowing for input source energy to be supplied from many sources such as a wind turbine, batteries, or a photovoltaic panel. Additionally the distiller may scale up to run on more conventional power supplies such as 110 Volt or 220 Volt systems.

The distiller according to the invention can be used to create a high quality water supply through a process of accelerated evaporation and condensation within a (typically) closed and controlled system. The distiller may be constructed in several ways allowing it to purify an existing water source or it may be reconfigured to condense water from atmospheric air. An embodiment of the invention keeps the system closed and purifies an existing source of water.

Rather than filtering water with conventional systems such as reverse osmosis or carbon filtration, the distiller according to the invention facilitates an evaporation and condensation process within what may be a closed system. This closed system creates an artificial environment within which the natural hydrologic cycle is dramatically accelerated so as to allow for pure water to be extracted from any source of water regardless of how contaminated it might be. The process is designed to be extremely efficient and reuses available energy within the distiller that conventional systems expel from the system. For example, excess heat energy created from the cooling system (in the condenser section) is used to heat the contaminated water source so as to accelerate the evaporation process and heat the air that is to carry the water through the system. It is essential that the air be warm enough to hold the water that is evaporating from the system so that the water can be carried to the cooling section and condensed back to a liquid form that is usable by the consumer.

Water is condensed within an environment within the distiller, that creates optimal conditions for evaporation with minimal energy requirements. In a typical distillation process water is heated to boiling point (approx 212 degrees). The water is then converted to steam and suspended in the air where it is moved to a cooling section that cools the air to below the dew point thus bringing the water back to a liquid state. This type of system is effective but has the need for considerable power in heating the water to such a high temperature. The distiller according to the invention offers a process with greater efficiency as water is heated with waste energy from the cooling system and is facilitated by numerous other means to ensure an efficient process. Once the water is evaporated and in a gaseous form, it may be filtered to remove contaminates. Any appropriate filter may be used for this purpose, such as a high quality HEPA filter, that ensures the air is pure and depleted of any contaminates that impede upon the quality of water created by the condensation process.

The moist hot air passes through the filter prior to entering a pre-cooling section of the device designed to increase efficiency. This section is essentially a dynamic heat pipe that moves heat from where it is not needed to where it is needed. This process pre-cools the air prior to the air entering the evaporator (cold) section of the cooling system where the water is forced to condense and is collected. The air then moves through the heat pipe again (upper portion) that may contain finned coils or other mechanism designed to provide adequate surface contact area, where the air is heated with the heat taken from the same air earlier in the lower section of the distiller. The air is then further heated by passing through the condenser section prior to the air being moved across the water evaporation section where the process begins again. In an alternative embodiment of the invention the external atmospheric air may be introduced into the system as a means to increase water output.

In addition to the benefits described above, the distiller may add additional value in further processing the water that is condensed so as to increase the value of the water. This process adds back the minerals in the water so as to accommodate the perceived value of these minerals by the consumer. However, the process may add organic minerals back into the water rather than inorganic minerals to ensure that the re-mineralization of the water is of real benefit to the human body, rather than simply adding back inorganic minerals that the human body cannot properly assimilate.

There are numerous means by which to put back minerals and trace elements into the water. This can be accomplished any number of ways but a design includes a small compartment with a hinged door allowing the door to be easily opened and closed. This compartment may be between the water storage container and the drip plate at the bottom of the evaporator (cooling element) so as to have all produced water pass through the chamber. The end user may insert into the chamber a mineral puck that fits into the provided space inside the compartment and as water drips over the puck the desired elements are added to the water.

Alternatively, the addition of elements (such as organic minerals) or the modification of the water source's properties may be done using an additional filter element that provides these elements or properties, or as part of an existing filter (for example as part of the activated carbon filter system).

Many consumers may want the addition of minerals or other beneficial elements added to the water, however as research indicates there is a strong argument both for, and against, adding minerals back into the water supply, the distiller allows the consumer the choice as to how they would like the drinking water treated.

In addition to adding back the mineral and trace elements expected to be in a typical healthy water source, this process may be used to add additional benefits to the water supply. Additional health remedies that could be added to the water include such things as colloidal silver, water oxygenation additives, negatively ionized hydrogen ions or other health enhancing products. With consumer awareness growing with regard to the health benefits offered by alkalized water and/or water that has antioxidant properties, these properties may be offered through the water source created by the system.

A distiller for purifying water is provided, including a condenser powered by an energy source; an evaporation pan for holding contaminated water; means for allowing heated air to enter the evaporation pan below the water, and entering an airflow above the evaporation pan; and an evaporator for collecting water from the airflow. The distiller may include a heat pipe to cool the airflow, after the airflow passes through the evaporator. The heat generated by the condenser may be used to heat the airflow before the airflow passes over the evaporation pan. A first fan may move the airflow and a second fan may control the heat generated by the condenser.

An evaporator is provided, including a housing for receiving airflow, the housing having an inner surface; a cooling element positioned within the housing in a loop-like fashion from approximately a first end of the housing to approximately a second end of the housing. The housing may have a slit along the length of the housing for receiving or expelling the airflow. The housing may be cylindrical and the cooling element coils may be near the inner surface of the housing, and the cooling element may not contact the inner surface.

An ultraviolet light bulb pass through the housing, the light bulb secured at the second end of the housing. A substantial portion of the inner surface may be layered with a reflective material, such as polished aluminium or polished stainless steel. The ultraviolet bulb may be retained by a housing bracket.

The cooling element may be coated with an antimicrobial material, such as AgION. A flow duct may be positioned at the first end or second end of the housing. A fan having a first setting may be used such that when on the first setting, airflow is drawn from the first end and at a second setting airflow is drawn from the slit.

A method of purifying water is provided, including (a) drawing airflow above an evaporation pan containing contaminated water; (b) passing the airflow through a housing, the housing having an inner surface and a cooling element shaped in a looped snake like fashion within the housing; (c) passing the airflow along the cooling element to exit the housing; (d) passing the airflow into a heat pipe; (e) passing the airflow through a condenser; and (f) passing the air through the evaporation pan; wherein water is collected from the airflow as the airflow passes through the housing. The housing may be cylindrical and the cooling element may be a coil. The airflow, after passing through the evaporation pan, may complete steps (a) through (f) again. The airflow may also pass through an air filter or a desiccant.

An evaporator is provided, including a housing having an air inlet and an air outlet; an ultraviolet light tube within the housing; a cooling element shaped in a plurality of loops around the ultraviolet light; wherein the housing encloses the ultraviolet light and the cooling element.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the numerous views, and wherein:

FIG. 1 is a block diagram of an embodiment of a distiller according to the invention showing the various parts in relation to each other;

FIG. 2 is a view of an embodiment of an evaporator used with the distiller; and

FIG. 3 is an exploded view of the internal components of such evaporator;

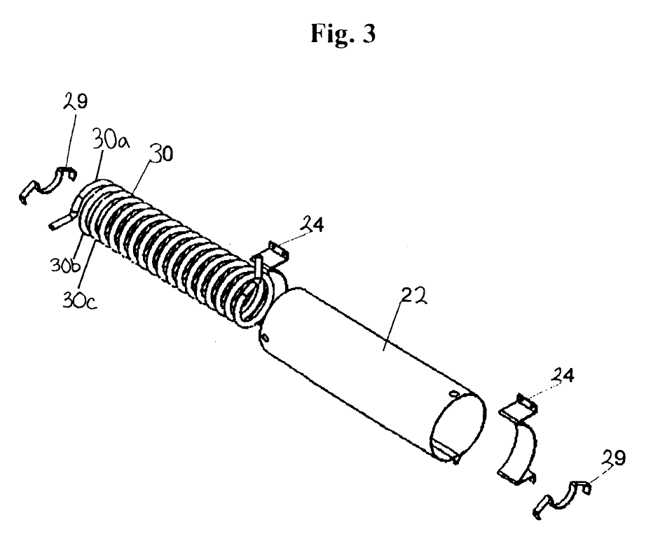

FIG. 4 is a view of an alternative embodiment of an evaporator used with the distiller;

FIG. 5 is an exploded view of the internal components thereof;

FIG. 6 is a block view showing an embodiment of a controller within a distiller according to the invention;

FIG. 7 is a flow chart showing the air passage within an embodiment of a distiller according to the invention; and

FIG. 8 is a flow chart showing the water flow within an embodiment of a distiller according to the invention.

### DETAILED DESCRIPTION OF THE INVENTION

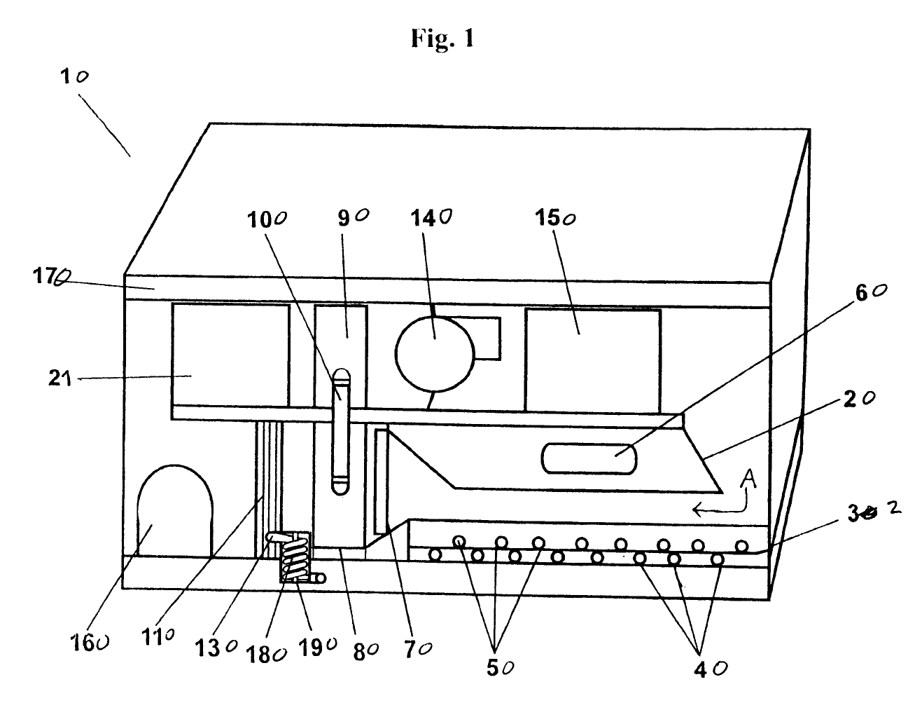

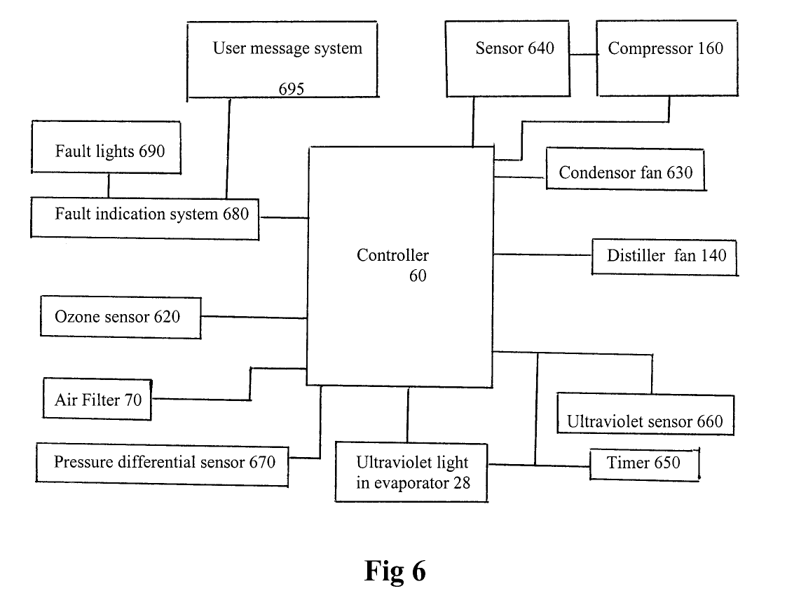

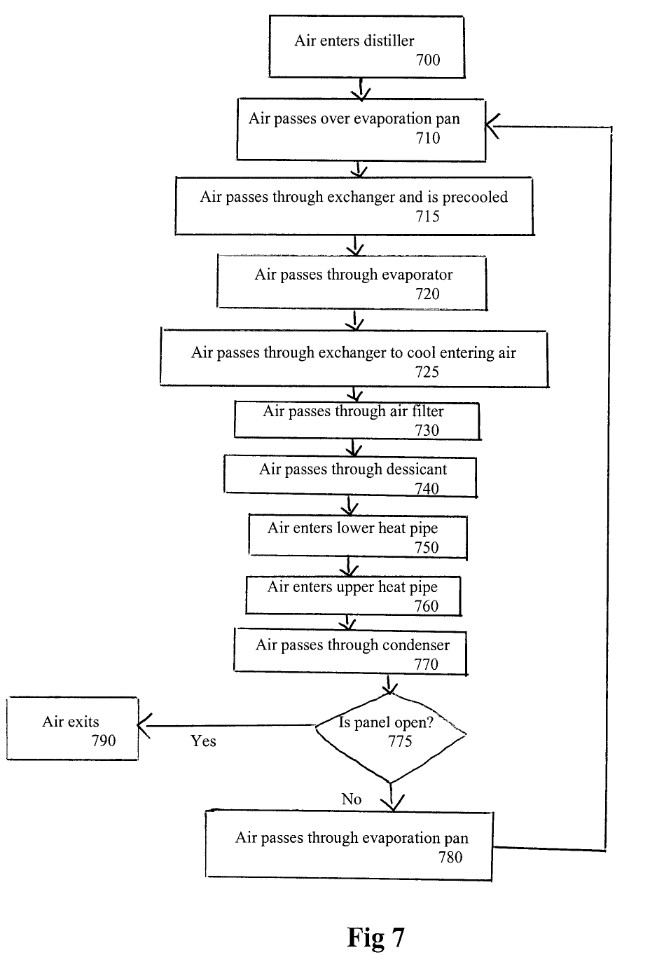

As shown in FIG. 1, distiller 10 has a closed system that uses the same air over and over throughout the purification process or may take in outside air to draw water from such air. FIG. 1 shows distiller 10 with the side panel removed exposing the internal parts of relevance. The following description should be read in conjunction with FIGS. 6, 7 and 8, which show elements of the controller system of distiller 10, the airflow thought distiller 10, and the water flow through distiller 10, respectively. Air enters the evaporation section of distiller 10 in direction A (step 700 as seen in FIG. 7). The air travels across evaporation pan 32 (step 710) where the air is saturated with moisture as a result of several processes. For example, the air is kept in close proximity to the water by airflow duct 20 and as the air passes over the water surface, the air continues to remove moisture from the air already positioned just above the water surface. The water in evaporation pan 32 may be contaminated or brackish water provided by the user or pumped in from an outside source. This allows for more molecules of water to enter the passing air and accelerates the evaporation process. Also, hot condenser pipes 40 carrying refrigerant (either gas or liquid) are situated just beneath evaporation pan 32. Such positioning assists in efficient operation of the cooling system by removing unwanted heat from that part of the cooling system, and also heating the water that is to be purified. This heating allows for the water to evaporate and be carried away by the air passing along the surface of the water. In addition to these systems, a feature of distiller 10 is that it provides some of the advantages of boiling without the need for high temperatures that are normally needed to create a boiling effect.

If distiller 10 is being used to extract water from atmospheric air, evaporation pan 32 would be empty.

In the embodiment shown in FIG. 1, artificial boiling pipes 50 create disturbances in the water that are normally associated with the boiling process, thus continuously breaking the surface tension of the water and facilitating an accelerated evaporation process. Air is pumped from within distiller 10 or externally, but warmer air is more effective, through numerous small apertures in artificial boiling pipes 50 that are situated at the bottom of evaporation pan 32. As air is pumped by air pump 60, bubbles form at the bottom of evaporation pan 32 and move to the surface where this highly moisture saturated air is carried away as the constant flow of air passes over the surface of the water.

While only one evaporator pan 32 is shown in FIG. 1, multiple evaporator pans may be used within the same distiller. For example a second evaporator pan may be situated just above the first with space for air to travel between the first and second evaporator pans 32 and the airflow may be divided such that half the air passes over the first evaporator pan and half the air passes over the second evaporator pan. This use of multiple evaporator pans 32 further increases the efficiency of the distiller. Each of evaporator pans 32 would have boiling pipes 50.

The air then passes through evaporator 21 (step 720). Evaporator 21 will now be explained in detail. Distiller 10 may incorporate any number of evaporators available, such as those using finned coils or tightly wound coils made of various materials. Examples of suitable evaporators 21 are described below.

A suitable embodiment of an evaporator 21 includes several benefits for the system, including self sterilization, reduced boundary layer build up, increased internal turbulence (i.e. surface area contact), and reduced resistance to airflow through distiller 10.

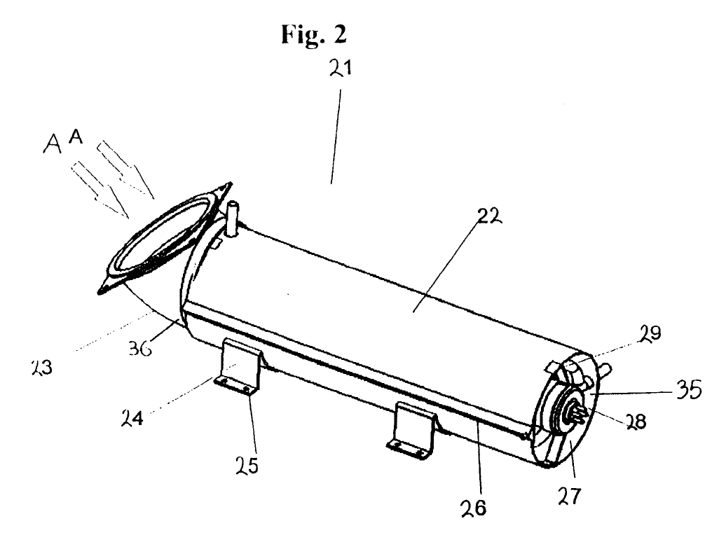

FIG. 2 shows an embodiment of an evaporator 21 for a water condensation and extraction apparatus, such as distiller 10. Evaporator 21 includes two main elements, a housing 22 through which the airflow travels, and cooling element 30 to accommodate the condensation and sterilization processes. Housing 22 may be air tight, and may be tube shaped to receive and hold cooling element 30 in place.

As air is drawn through housing 22 via a fan 140, into evaporator 21, the airflow is exposed to turbulence, while at the same time cooling element 30 provides a reduced resistance to the airflow. As the airflow flows into housing 22 in direction AA, the air passes through the space between UV bulb 28 (that may or may not generate ozone), and the inner wall 27 of housing 22. Inner surface 27 of evaporator 21 is highly reflective and may be made of one or more materials that reflect UV light, such as a highly polished aluminium or stainless steel. Light radiating from UV bulb 28, which is held in place with housing bracket 29, disinfects the surfaces of cooling element 30. In an embodiment of the invention, cooling element 30 is a series of loops forming a coil situated between the inner surface 27 of housing 22, and UV bulb 28, but contacting neither housing 22 or UV bulb 28, and is made of a material such as copper or stainless steel for the desired properties and material cost.

Cooling element 30 may be coated with a material such as AgION, that offers additional antimicrobial properties to assist in keeping cooling element 30 clean and free of contaminates. Additionally, other surfaces of evaporator 21, such as inner wall 27, that are exposed to either the airflow or the produced water, may be coated with such a material. These materials (such as AgION) also provide anticorrosive properties and desirable thermal properties. Some materials that may be used in the manufacture of evaporator 21 include Eldon James antimicrobial Flexelene™ Silver, CPT-324 Polyaspartic NSF Coating, ControlTech™ and Tank Clad™ made by the Sherwin-Williams Co., SilverSan™ Antimicrobial Powder Coating, amongst others.

As the airflow is drawn through evaporator 21, it comes in contact with the first coil turn 30a, where the air is drawn around the coil, and directed to contact with the next adjacent coil 30b. The air is then be drawn around coil 30b and directed toward next coil 30c and so on down the length of cooling element 30. This action increases the turbulence of the airflow and reduces the unwanted boundary layer caused when air passes through a series of thin fins, as a layer (the boundary layer) is typically built between the airflow and the contact surface. This boundary layer allows air to pass through the evaporator without being directly exposed to a contact surface, and thus allows the air to pass through the evaporator while maintaining an undesirably high moisture level.

The incoming airflow may enter evaporator 21 from either back end 35 or front end 36. While the recommended location for using distiller 10 is outside, so that outside air can enter, this may not be advisable for a variety of reasons. For example, if distiller 10 is used in an environment where theft is a concern the consumer may want to locate the unit inside a dwelling. In such a case, it remains advisable that distiller 10, and evaporator 21 access incoming airflow from an outside source (if the user is using distiller 10 to extract water from atmospheric air) of internally (if the user is using distiller 10 to purify contaminated water). Movable flow duct 23 may be positioned at front end 36, as shown in FIG. 2, or may be positioned to draw air from back end 35 of evaporator 21. The movable flow duct 23 may be positioned to face any direction so as to accommodate a variety of internal designs and applications. Evaporator 21 may be mounted within distiller 10 with mounting means such as bracket 24 used with a fastening mechanism, such as screws, nails or glue (apertures 25 for screws are shown in FIG. 2). Alternatively, evaporator 21 could be used as part of an alternate embodiment, and be used outside of distiller 10 for an alternative function. To facilitate manufacture of evaporator 21, housing 22 may be provided as a single tube shaped component, or in flat sections that may be rolled up and secured with latching seam 26 of some kind (such as used in conventional aluminium ducting). Housing 22 may be shaped to provide a channel at its lowest point, to aid the movement of water therein.

The internal function of evaporator 21 may provide sterilization unattainable by conventional evaporators. The sterilization mechanism as described above offers UV and/or ozone sterilization for the internal parts of evaporator 21 (including the shaded side of the coils), as well distiller 10's other internal parts that are exposed to either water or air. UV light 28 may be an ozone producing light, so that when distiller 10 is shut down, controller 600, as seen in FIG. 6, at preset time intervals, will activate ozone producing UV light 28 to disinfect evaporator 21 with the UV light it creates. As there is an accumulation of ozone inside evaporator 21, once the ozone is at an appropriate level again as determined by controller 600, drive fan 140 moves the ozone saturated air in a first direction (such as reverse of direction AA) to disinfect all contact surfaces in front of evaporator 21 including any intake air filters. When adequate time has passed (according to timer 650), the fan is turned off and again ozone levels elevate in evaporator 21. Once the desired ozone levels are reached, which can be determined by any number of sensing methods, including the allotment of time for accumulation or ozone sensor 620), fan 140 turns on in the direction opposite the preceding direction (in this example forward in direction AA), to allow the ozone saturated air to pass though distiller 10 beyond evaporator 21 to keep the parts of distiller 10 so positioned clean as well.

In an alternative embodiment of the invention, housing 22 need not be cylindrical and could be an alternative shape, such as a cuboid, or toroid. In such a case, cooling element 30 would be positioned near the inner surface 27 and extend in a snake-like fashion to form loops (circular or otherwise) from the first end of the cuboid to the second end thereof.

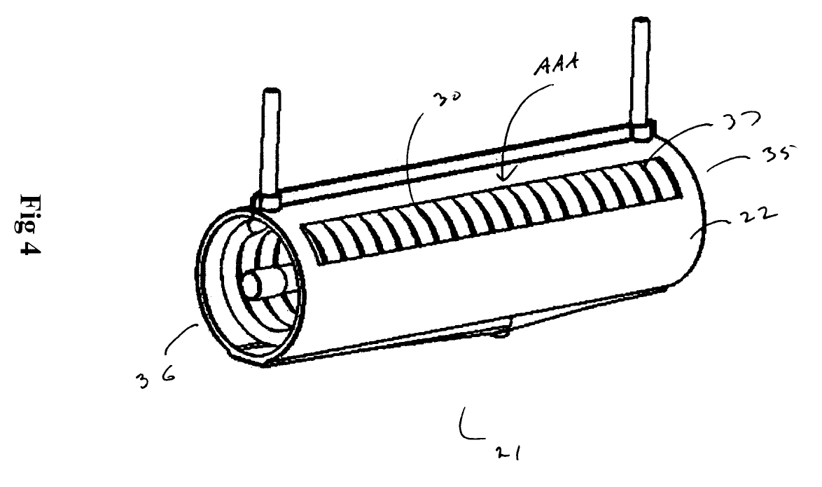

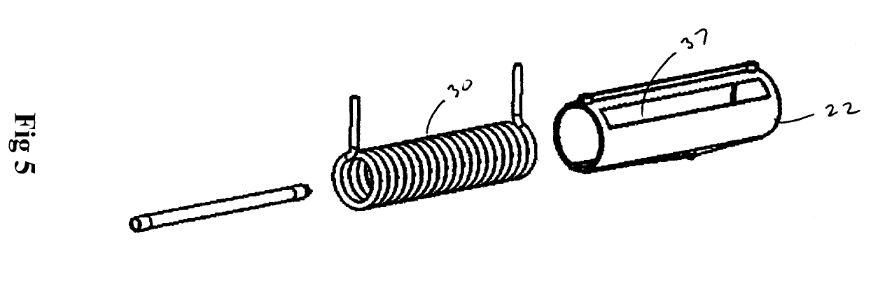

In an alternative embodiment of evaporator 21, as seen in FIGS. 4 and 5, air may enter or be extracted from (as described above) housing 22 from the side of housing 22 through slit 37 rather than the ends 35, 36 thereof. In the previous embodiment, where air enters in from one end of housing 22 and travels through to the other end, the first turns of coil 30 have the most effect in reducing the air temperature and condensing water, so that when the air gets to later coils, the air has already been significantly depleted of its moisture, rendering those later coil turns less effective.

In the “cross flow” air intake design as seen in FIGS. 4 and 5, in the case where air enters into housing 22 in direction AAA, through slit 37 that is positioned along the length of housing 22. This air may leave housing 22 from either front end 36 or back end 35 of housing 22, allowing more of coils 30 to be used in cooling the air and condensing water.

Distiller 10 may utilize any number of filters to ensure that the water that is condensed maintains purity. In FIG. 1 only one air filter 70 is shown and it is situated between the evaporator 21 and the lower heat pipe 80. Numerous different types of air filters 70 could be used such as an electrostatic filter, and/or other filters that may be cleaned and reused. Once a suitable air filter 70 such as a HEPA filter cleans the air (step 730), the air enters the pre-cooling device, lower heat pipe 80 (step 750).

Optionally, a desiccant (not shown) may be also used (step 740). This desiccant may be in virtually any form, however an embodiment could be a desiccant wheel such as is currently used in a variety of devices designed for the extraction of moisture from air. This desiccant could be situated anywhere along the airflow path, but an optimal location is immediately after air filter 70.

In lower heat pipe 80, the air is cooled as the heat pipe drives heat upward to upper heat pipe 90. This heat pipe system is designed to use heat pipe technology that does not require regeneration and allows for a constant flow of refrigerant through the system.

Both the upper heat pipe 90 and lower heat pipe 80 may use typical cooling fins as are normally seen with conventional evaporators and condensers for air conditioners in order to ensure maximum surface area is available for the air to contact. As the airflow system in distiller 10 is designed to be continuous and not require down time for regeneration, a complete circuit of refrigerant flows through the heat pipe sections 80, 90. Refrigerant connector pipes 100 may be situated on either side of upper heat pipe 90 and lower heat pipe 80 allowing for the refrigerant to move easily from the lower heat pipe 80 to upper heat pipe 90, and then back to the lower heat pipe 80 again in a continuous flow so as not to require a regeneration cycle.

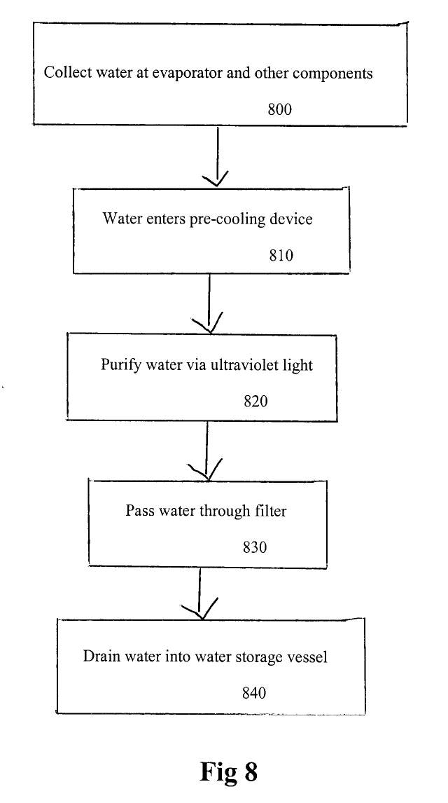

Once the air has past through the pre-cooling section of lower heat pipe 80 it may then pass through a secondary pre-cooling device 110 (step 760) that provides cold water moving through it that is provided by evaporator 21 as evaporator 21 cools the air passing through the system to below dew point thereby condensing that water from the air. Water that is collected at evaporator 21 (step 800 in FIG. 8) may drain directly out of distiller 10 (step 840) or it may move through a secondary pre-cooling device 110 fitted with a water drain 130 (step 810). In the embodiment shown in FIG. 1, water drains out of evaporator 21 through water drain 130 however distiller 10 may have means to capture condensed water from numerous locations where it may be produced, such as lower heap pipe 80, which also has the capacity to draw some water from the air. Water may also be produced at secondary pre-cooling device 110, and evaporator 21 (where most of the water will be produced).

Once the cold air passes through evaporator 21 and lower heat pipe 80, and as much water has been condensed as possible from the air, the air passes through upper heat pipe 90 so as to remove the heat that was drawn away from the air as it passed through lower heat pipe 80 (step 760). This allows for efficient processing of air through distiller 10. An alternative embodiment of distiller 10 may have an air to air heat exchanger configured such that before the air leaves evaporator 21 it passes through the exchanger (step 725) such that the air entering the evaporator 21 is pre-cooled (step 715) thus using the cold created by distiller 10 to pre-cool the air that is about to enter evaporator 21.

Once the air has passed through the upper heat pipe 90 it will have taken heat away from the heat pipe system 80, 90 allowing heat pipe 80, 90 to operate more effectively. In addition, the air will have been heated to some degree and as only warm air can hold significant amounts of water, the heating of this air is necessary if the air is to effectively pick up the moisture when it passes through evaporation pan 32. At some point in the system the air passes through drive fan 140 that circulates the air through distiller 10. Drive fan 140 may be situated literally anywhere in distiller 10 that is convenient. Drive fan 140 may be controlled such that it creates the optimal airflow through distiller 10, and can be controlled automatically or manually so as to create optimal airflow. In addition to the airflow being controlled, the cooling system may be controlled, as described below, either manually or automatically to ensure optimal operation.

In addition to fan 140 that controls the airflow through evaporator 21, distiller 10 may incorporate a controllable fan 630 for condenser 150. This fan (or fans) control how much heat is being taken away from distiller 10 and can be used to broaden the efficient operating range of distiller 10. Metering of refrigerant assists in controlling the cooling process, but control over the airflow passing through condenser 150 assists in balancing the refrigerant pressures even under varying loads and improves overall performance of distiller 10.

Air that has passed through upper heat pipe 90 will then pass through condenser 150 (step 770) where the air will cool condenser 150 and thus assist in the efficient function of the cooling system. As well, condenser 150 further heats the air as is needed for the air to be ready to again pass through evaporation pan 32 (step 780). Air traps may be used to ensure water does not enter boiling pipes 50 when distiller 10 is “off”, although air entering boiling pipes 50 would be expelled when distiller 10 is restarted. Air moves around and under airflow duct 20, keeping the air in close proximity to the surface of the hot water that is being disturbed by the artificial boiling process created by the system (step 710). This process of continuously breaking the surface tension of the water while air is continuously drawing away moisture allows for an evaporation process that is akin to boiling but without the energy requirements of boiling. The cooling system's compressor 160 may be kept in the system, ideally where the air is to be heated, such as just before or after condenser 150 to assist in heating the air, or compressor 160 may be isolated outside the airflow system. Condenser 150 may be mounted outside of distiller 10 to remove excessive heat.

If distiller 10 is being used to purify existing water, the airflow may be a closed system, so that exterior air is not needed. If distiller 10 us being used to extract water from atmospheric air, air must be obtained from outside distiller 10 and evaporation pan 32 would be empty. For such a use, a panel (not shown) in distiller 10 is opened (step 775), to allow the airflow to escape after passing through condenser 150 (step 790). Baffles may be used to ensure air expelled from distiller 10 does not re-enter the system.

Controller 600 in distiller 10 may be used to ensure compressor 160 does not overheat. Controller 600 receives input from sensor 640 about the temperature of compressor 160, and when the controller recognizes that compressor 160 has reached a maximum temperature, the controller shuts down compressor 160 allowing adequate time for it to cool before restarting distiller 10.

Compressor 160 may be a 12 Volt compressor that may be portable or allow for input source energy to be supplied from many sources such as a wind turbine, batteries, or a photovoltaic panel. Alternatively, distiller 10 may be scaled up to operate on more conventional power supplies such as 110 Volt or 220 Volt systems.

Insulation 170 may be used to keep the heat energy where it is needed or alternatively, cooling fins exposed to the outside air around distiller 10 may be used in parts of distiller 10 where a cooling effect is desirable, such as just prior to evaporator 21 (or for example, in the cooling section such as heat pipe 80, 90, and condenser 150).

The water that is collected from distiller 10 (step 800 in FIG. 8) may be exposed to various means to ensure only high quality of water is being created. Ultraviolet lights 190 and/or water purification systems (not shown) such as reverse osmosis or carbon filtration systems may be used.

As water is condensed, gravity draws the water down to a storage tank (not shown) preferably just beneath distiller 10. The water outtake may be fitted with a p-trap to ensure outside elements are not able to negatively impact the internal mechanisms of the system. As well, the water may be exposed to ultraviolet purification just prior to leaving distiller 10 (step 820), after the air is used in pre-cooling device 110. Optionally, especially where there is a storage vessel directly below distiller 10, the water may pass through a light exposure tube 180 that is transparent to ultraviolet light (quartz or Teflon). The water in the storage vessel can be circulated again through any or all elements of distiller 10 so as to ensure the water is kept clean of unwanted contaminates and does not stagnate. One embodiment, as shown in FIG. 1, would be an ultraviolet light 190 that has a light exposure tube 180 wrapped around it thus allowing considerable time for the water to be in close proximity to the light as gravity draws it in a spiral formation around the ultraviolet light 190 prior to the water leaving distiller 10.

Distiller 10 may be fitted with a device (for example, a simple switch) that provides that if air filter 70 is not installed, controller 600 will not allow distiller 10 tot operate. In addition, distiller 10 may be designed such that if any of the critical components are either not installed or not working properly, distiller 10 will not operate. This can be accomplished with sensor mechanisms and a switching system working with controller 600.

With the proper use of ultraviolet light 99.99% of all algae, bacteria and/or viruses may be killed. One or more ultraviolet lights 190 may be installed in distiller 10 at the water drain. Ultraviolet light wavelengths can range from 180 to 480 nanometers, but the band of light that is attributed with the greatest sterilization properties is much narrower; typically between 250 and 260 nanometers. Ultraviolet light tends to degrade in intensity and wavelength over time and as well the ultraviolet light may solarize, or darken the quartz glass material, further reducing the transmission of the desired light properties. As well, the intermittent use of an ultraviolet light may further reduce its sterilizing properties. Therefore, while an ultraviolet light may still be illuminated, the desired properties of the light may no longer be present.

Ultraviolet light 190 may be on timer 650 that indicates when light 190 should be changed, based on hours of operation, regardless of whether or not it the light 190 still illuminates. In an alternative embodiment, an ultraviolet sensor 660 may be incorporated into the device such that when the desired wavelength is no longer present controller 600 will indicate this to the user by use of a message or indicator, or simply turn distiller 10 off.

As quartz is transparent to ultraviolet light, it may be used to separate the ultraviolet light from the water, or the water may pass directly over the casing of ultraviolet light 190. These waves with the germicidal properties that are desired can penetrate Teflon without the negative effects of accumulation of film on those components of distiller 10 exposed to this form of light. Teflon thus may be used in any section of distiller 10 that is exposed to this type of light.

The condition of air filter 70 may also be monitored in a number of ways. For example, a pressure differential sensor 670 may provide controller 600 an indication of the state of filter 70, however numerous other design applications may be used for this purpose, including monitoring the state of fan 140 to determine how much resistance is being created by the airflow in distiller 10, or using timer 650 to indicate when air filter 70 needs to be replaced.

Distiller 10 may also be fitted with a water filter (not shown) in addition to the other systems designed to ensure that the water produced by distiller 10 is of high quality (i.e. air filter and ultraviolet lighting systems). This water filter may be placed just below the drainage spout of the condenser unit (after or before the ultraviolet light) or anywhere in the line between the evaporator 21 and the storage vessel or at/in the storage vessel. The water purification system may be carbon, reverse osmosis, various membrane systems, etc. Thus water may pass through the water filter (step 830) just prior to leaving distiller 10, and entering water storage vessel (step 840).

An embodiment of distiller 10 includes a fault indication system 680 that has one or more LED lights 690 viewable by the user that indicate when there is a maintenance issue. One light 690, or one color of light, could be used to indicate that ultraviolet light 190 is not working and another light 690 (or an alternate color of the same light) could be used to indicate that filter 70 needs to be changed. If filter 70 is not changed, or if ultraviolet light 190 is not working, distiller 10 may be set up to shut itself off until the appropriate action is taken. Such a fault indication system 680 may provide, a fault light 690 in the front of a panel on distiller 10 lights, and that when such faults occurs, including air filter 70 being plugged, compressor 150 overheating, or ultraviolet light 180 burning out, distiller 10 stops functioning. To locate the fault, a user can push a “start” button visible on a control panel (not shown) to activate a user message system 695, which will play an audio message, in one of a number of languages. To correct the fault, the user removes the cover (not shown) of distiller 10, makes the necessary repair and presses a “reset button” (not shown) inside distiller 10. When the cover is mounted, distiller 10 can be restarted by pushing the “start” button. Note distiller 10 cannot be restarted until the cover is replaced, as it should include a power disconnect when the cover is removed to allow safe access to the components of distiller 10.

Although the particular preferred embodiments of the invention have been disclosed in detail for illustrative purposes, it will be recognized that variations or modifications of the disclosed apparatus lie within the scope of the present invention.

We claim:

1. A distiller for purifying water, comprising:
(a) a condenser powered by an energy source;
(b) an evaporation pan for holding contaminated water;
(c) means for allowing heated air to enter said evaporation pan below said water, and entering an airflow above said evaporation pan; and
(d) an evaporator for collecting water from said airflow.

2. The distiller of claim 1 further comprising a heat pipe to cool said airflow, after said airflow passes through said evaporator.

3. The distiller of claim 2 wherein the heat generated by said condenser is used to heat said airflow before said airflow passes over said evaporation pan.

4. The distiller of claim 1 further comprising a fan for moving said airflow.

5. The distiller of claim 4 further comprising a second controllable fan for controlling heat generated by said condenser.

6. An evaporator comprising:
a) a housing for receiving airflow, said housing having an inner surface; and
b) a cooling element, said cooling element positioned within said housing in a loop-like fashion from approximately a first end of said housing to approximately a second end of said housing.

7. The evaporator of claim 6 wherein said housing further comprises a slit along the length of said housing for receiving or expelling said airflow.

8. The evaporator of claim 7 wherein said housing is cylindrical, and said cooling element coils near the inner surface of said housing, and said cooling element does not contact said inner surface.

9. The evaporator of claim 8 wherein an ultraviolet light bulb passes through said housing, said light bulb secured at said second end of said housing.

10. The evaporator of claim 9 wherein a substantial portion of said inner surface is layered with a reflective material.

11. The evaporator of claim 10 wherein said reflective material is polished aluminium.

12. The evaporator of claim 10 wherein said reflective material is polished stainless steel.

13. The evaporator of claim 9 wherein said ultraviolet bulb is retained by a housing bracket.

14. The evaporator of claim 10 wherein said cooling element is coated with an antimicrobial material.

15. The evaporator of claim 14 wherein said antimicrobial material is AgION.

16. The evaporator of claim 14 wherein a flow duct is positioned at said first end.

17. The evaporator of claim 14 wherein a flow duct is positioned at said second end.

18. The evaporator of claim 16 further comprising a fan having a first setting, and when on said first setting, draws said airflow from said first end.

19. The evaporator of claim 18 wherein said fan has a second setting, and when on said second setting draws airflow from said slit.

20. A method of purifying water, comprising:
(a) drawing airflow above an evaporation pan containing contaminated water
(b) passing said airflow through a housing, said housing having an inner surface and a cooling element shaped in a looped snake like fashion within said housing;
(c) passing said airflow along said cooling element to exit said housing;
(d) passing said airflow into a heat pipe;
(e) passing said airflow through a condenser; and
(f) passing said air through said evaporation pan;
wherein water is collected from said airflow as said airflow passes through said housing.

21. The method of claim 20 wherein said housing is cylindrical and said cooling element is a coil.

22. The method of claim 20 wherein said airflow, after passing through said evaporation pan, complete steps (a) through (f) again.

23. The method of claim 20 wherein said airflow further passes through an air filter.

24. The method of claim 20 wherein said airflow passes through a desiccant.

25. An evaporator, comprising:
(a) a housing having an air inlet and an air outlet;
(b) an ultraviolet light tube within said housing;
(c) a cooling element shaped in a plurality of loops around said ultraviolet light; and
wherein said housing encloses said ultraviolet light and said cooling element.
